SEQUENCE LISTING (4 sequences)

SEQ ID NO: 1 - DNA, 1419 bp, Artificial Sequence
  Synthetic construct tetx gene for Hc fragment of tetanus toxin
atgggcagcagccatcatcatcatcatcacagcagcggcctggtgccgcgcggcagccatatgaaaaaccttgattgttgggtcgacaacgaagaagacatcgatgttatcctgaaaaagtctaccattctgaacttggacatcaacaacgatattatctccgacatctctggtttcaactcctctgttatcacatatccagatgctcaattggtgccgggcatcaacggcaaagctatccacctggttaacaacgaatcttctgaagttatcgtgcacaaggccatggacatcgaatacaacgacatgttcaacaacttcaccgttagcttctggctgcgcgttccgaaagtttctgcttcccacctggaacagtacggcactaacgagtactccatcatcagctctatgaagaaacactccctgtccatcggctctggttggtctgtttccctgaagggtaacaacctgatctggactctgaaagactccgcgggcgaagttcgtcagatcactttccgcgacctgccggacaagttcaacgcgtacctggctaacaaatgggttttcatcactatcactaacgatcgtctgtcttctgctaacctgtacatcaacggcgttctgatgggctccgctgaaatcactggtctgggcgctatccgtgaggacaacaacatcactcttaagctggaccgttgcaacaacaacaaccagtacgtatccatcgacaagttccgtatcttctgcaaagcactgaacccgaaagagatcgaaaaactgtataccagctacctgtctatcaccttcctgcgtgacttctggggtaacccgctgcgttacgacaccgaatattacctgatcccggtagcttctagctctaaagacgttcagctgaaaaacatcactgactacatgtacctgaccaacgcgccgtcctacactaacggtaaactgaacatctactaccgacgtctgtacaacggcctgaaattcatcatcaaacgctacactccgaacaacgaaatcgattctttcgttaaatctggtgacttcatcaaactgtacgtttcttacaacaacaacgaacacatcgttggttacccgaaagacggtaacgctttcaacaacctggacagaattctgcgtgttggttacaacgctccgggtatcccgctgtacaaaaaaatggaagctgttaaactgcgtgacctgaaaacctactctgttcagctgaaactgtacgacgacaaaaacgcttctctgggtctggttggtacccacaacggtcagatcggtaacgacccgaaccgtgacatcctgatcgcttctaactggtacttcaaccacctgaaagacaaaatcctgggttgcgactggtacttcgttccgaccgatgaaggttggaccaacgactaa

SEQ ID NO: 2 - protein, 472 aa, Artificial Sequence
  Hc fragment of tetanus toxin
MGSSHHHHHHSSGLVPRGSHMKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 3 - DNA, 4338 bp, Clostridium tetani
gcttcatatgagtcgtcaagctgtatataaaaataaggttttagcattaaaaaaattagaacctatagtaaataaattaattaatatatagtttttataatttaattatgaataatattcttaagataaaaagtaaatttttaaaaatttaaattttcagtttacaaaaaataacctgattatgttatatgtaattgtaaaaaacatataaaaaatcagaaaaatttaggaggtatattattaatggattaaataataattttttaatttacttttgattaataaatattaaatgtttattttaattaggagatgatacgtatgccaataaccataaataattttagatatagtgatcctgttaataatgatacaattattatgatggagccaccatactgtaagggtctagatatctattataaggctttcaaaataacagatcgtatttggatagtgccggaaaggtatgaatttgggacaaaacctgaagattttaacccaccatcttcattaatagaaggtgcatctgagtattacgatccaaattatttaaggactgattctgataaagatagatttttacaaaccatggtaaaactgtttaacagaattaaaaacaatgtagcaggtgaagccttattagataagataataaatgccataccttaccttggaaattcatattccttactagacaagtttgatacaaactctaattcagtatcttttaatttattagaacaagaccccagtggagcaactacaaaatcagcaatgctgacaaatttaataatatttggacctgggcctgttttaaataaaaatgaggttagaggtattgtattgagggtagataataaaaattacttcccatgtagagatggttttggctcaataatgcaaatggcattttgcccagaatatgtacctacctttgataatgtaatagaaaatattacgtcactcactattggcaaaagcaaatattttcaagatccagcattactattaatgcacgaacttatacatgtactacatggtttatacggaatgcaggtatcaagccatgaaattattccatccaaacaagaaatttatatgcagcatacatatccaataagtgctgaagaactattcacttttggcggacaggatgctaatcttataagtattgatataaaaaacgatttatatgaaaaaactttaaatgattataaagctatagctaacaaacttagtcaagtcactagctgcaatgatcccaacattgatattgatagctacaaacaaatatatcaacaaaaatatcaattcgataaagatagcaatggacaatatattgtaaatgaggataaatttcagatactatataatagcataatgtatggttttacagagattgaattgggaaaaaaatttaatataaaaactagactttcttattttagtatgaatcatgaccctgtaaaaattccaaatttattagatgatacaatttacaatgatacagaaggatttaatatagaaagcaaagatctgaaatctgaatataaaggacaaaatatgagggtaaatacaaatgcttttagaaatgttgatggatcaggcctagtttcaaaacttattggcttatgtaaaaaaattataccaccaacaaatataagagaaaatttatataatagaactgcatcattaacagatttaggaggagaattatgtataaaaattaaaaatgaagatttaacttttatagctgaaaaaaatagcttttcagaagaaccatttcaagatgaaatagttagttataatacaaaaaataaaccattaaattttaattattcgctagataaaattattgtagattataatctacaaagtaaaattacattacctaatgataggacaaccccagttacaaaaggaattccatatgctccagaatataaaagtaatgctgcaagtacaatagaaatacataatattgatgacaatacaatatatcaatatttgtatgctcaaaaatctcctacaactctacaaagaataactatgactaattctgttgatgacgcattaataaattccaccaaaatatattcatattttccatctgtaatcagtaaagttaaccaaggtgcacaaggaattttattcttacagtgggtgagagatataattgatgattttaccaatgaatcttcacaaaaaactactattgataaaatttcagatgtatccactattgttccttatataggacccgcattaaacattgtaaaacaaggctatgagggaaactttataggcgctttagaaactaccggagtggttttattattagaatatattccagaaattactttaccagtaattgcagctttatctatagcagaaagtagcacacaaaaagaaaagataataaaaacaatagataactttttagaaaaaagatatgaaaaatggattgaagtatataaactagtaaaagcaaaatggttaggcacagttaatacgcaattccaaaaaagaagttatcaaatgtatagatctttagaatatcaagtagatgcaataaaaaaaataatagactatgaatataaaatatattcaggacctgataaggaacaaattgccgacgaaattaataatctgaaaaacaaacttgaagaaaaggctaataaagcaatgataaacataaatatatttatgagggaaagttctagatcatttttagttaatcaaatgattaacgaagctaaaaagcagttattagagtttgatactcaaagcaaaaatattttaatgcagtatataaaagcaaattctaaatttataggtataactgaactaaaaaaattagaatcaaaaataaacaaagttttttcaacaccaattccattttcttattctaaaaatctggattgttgggttgataatgaagaagatatagatgttatattaaaaaagagtacaattttaaatttagatattaataatgatattatatcagatatatctgggtttaattcatctgtaataacatatccagatgctcaattggtgcccggaataaatggcaaagcaatacatttagtaaacaatgaatcttctgaagttatagtgcataaagctatggatattgaatataatgatatgtttaataattttaccgttagcttttggttgagggttcctaaagtatctgctagtcatttagaacaatatggcacaaatgagtattcaataattagctctatgaaaaaacatagtctatcaataggatctggttggagtgtatcacttaaaggtaataacttaatatggactttaaaagattccgcgggagaagttagacaaataacttttagggatttacctgataaatttaatgcttatttagcaaataaatgggtttttataactattactaatgatagattatcttctgctaatttgtatataaatggagtacttatgggaagtgcagaaattactggtttaggagctattagagaggataataatataacattaaaactagatagatgtaataataataatcaatacgtttctattgataaatttaggatattttgcaaagcattaaatccaaaagagattgaaaaattatacacaagttatttatctataacctttttaagagacttctggggaaaccctttacgatatgatacagaatattatttaataccagtagcttctagttctaaagatgttcaattgaaaaatataacagattatatgtatttgacaaatgcgccatcgtatactaacggaaaattgaatatatattatagaaggttatataatggactaaaatttattataaaaagatatacacctaataatgaaatagattcttttgttaaatcaggtgattttattaaattatatgtatcatataacaataatgagcacattgtaggttatccgaaagatggaaatgcctttaataatcttgatagaattctaagagtaggttataatgccccaggtatccctctttataaaaaaatggaagcagtaaaattgcgtgatttaaaaacctattctgtacaacttaaattatatgatgataaaaatgcatctttaggactagtaggtacccataatggtcaaataggcaacgatccaaatagggatatattaattgcaagcaactggtactttaatcatttaaaagataaaattttaggatgtgattggtactttgtacctacagatgaaggatggacaaatgattaaacagattgatatgttcatgattactctatataaaaaattaaataatataacaatctagctatattattt

SEQ ID NO: 4 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

(19) United States
(12) Patent Application Publication
(10) Pub. No.: US 2018/0228925 A1
(43) Pub. Date: Aug. 16, 2018

(54) Quantifying Net Axonal Transport in Motor Neuron Pathologies

(71) Applicant: University of Massachusetts, Boston, MA (US)

(72) Inventors: Robert H. Brown, JR., Needham, MA (US); Justin Pin-Tsun Lee, Boston, MA (US); Mary Rusckowski, Southborough, MA (US)

(21) Appl. No.: 15/752,611

(22) PCT Filed: Aug. 26, 2016
(86) PCT No.: PCT/US16/49103

§ 371 Date: Feb. 14, 2018

Related U.S. Application Data
(60) Provisional application No. 62/211,526, filed on Aug. 28, 2015.

(51) Int. Cl. A61K 51/08; A61K 35/28; A61B 6/03; A61B 5/055; A61B 5/04; A61B 5/00

(52) U.S. Cl. CPC A61K 51/088; A61K 35/28; A61B 6/032; A61B 5/055; A61B 5/4041; A61B 5/0071; A61B 6/037; A61B 5/4082; A61B 5/04001

(57) ABSTRACT

Methods of monitoring and diagnosing subjects with motor neuron pathology such as motor neuron disorders (including but not limited to amyotrophic lateral sclerosis (ALS)) and neuropathies, based on imaging of a labeled fragment of tetanus toxin, e.g., tetanus toxin C fragment.

### CLAIM OF PRIORITY

This application claims the benefit of U.S. Provisional Patent Application Ser. No. 62/211,526, filed on Aug. 28, 2015. The entire contents of the foregoing are hereby incorporated by reference.

### TECHNICAL FIELD

Described herein are methods of monitoring and diagnosing subjects with motor neuron pathology such as motor neuron disorders (including but not limited to amyotrophic lateral sclerosis (ALS)) and neuropathies, based on imaging of a labeled fragment of tetanus toxin, e.g., tetanus toxin C fragment.

### BACKGROUND

A wide variety of disorders adversely affect motor neurons, leading to weakness or outright paralysis of the muscles the neurons innervate. Examples of these pathologies include amyotrophic lateral sclerosis (ALS, or Lou Gehrig's disease), peripheral neuropathies (numerous subtypes), and traumatic injuries to nerve.

### SUMMARY

Described herein are methods to record, in vivo in living small and large mammals, how rapidly a molecular cargo is transported from a muscle to the cell body of motor neurons that innervate that muscle; the method applies to motor neurons in the spinal cord and brainstem. The transport rate up the motor nerve to the cell body (so-called retrograde axonal transport) is a measure of viability of the motor neuron and the integrity of the neuromuscular junction. Because the method described herein quantitates transport across the neuromuscular junction as well as retrograde transport up the axon it is referred to herein as “net axonal transport”. This parameter can be used to characterize the status of the motor nerve, potentially assisting in diagnosing various motor neuron pathologies like amyotrophic lateral sclerosis, peripheral neuropathy or traumatic nerve injury. This method provides a quantitative biomarker for motor neuron function that can also be used for monitoring disease progress or benefit of therapies for these pathologies. These biomarkers can also be used to facilitate study of the biology and treatment of these conditions.

Thus, in a first aspect the invention provides methods for diagnosing pathology in motor neurons, e.g., a motor neuron pathology or neuropathy, in a subject. The methods include locally administering to a muscle of the subject a radiolabeled agent comprising tetanus toxic C fragment; obtaining a first image of the agent in the subject prior to the muscle injection at a first time point and subsequently obtaining at least a second image of the agent in the subject at a subsequent time point and then optionally multiple additional images at later time points; determining at each time point after injection the total amount of the agent that is taken up into the cell bodies of the motor neurons that innervate the injected muscle, to determine net total transport at that time point; and using the net total transport at the different time points to calculate the rate of uptake of the radiolabeled agent in cell bodies of the motor neurons over time, to determine net total axonal transport rate; and diagnosing the subject as having a motor neuron pathology, e.g., a motor neuron disease or neuropathy, when the net total transport and net axonal transport rate are reduced relative to reference net total transport and net axonal transport rate.

In some embodiments, the methods include selecting and/or administering a treatment for a motor neuron pathology to the subject, e.g., riluzole, radicut, Edaravone, inosine, or stem cells.

Also provided herein are methods for monitoring a motor neuron pathology in a subject. The methods include determining net total transport and/or net axonal transport rate in the subject at a first time point; determining net total transport and/or net axonal transport rate in the same subject at a second time point; comparing net total transport and/or net axonal transport rate in the subject at the first and second time points; wherein a reduction in net total transport or net total axonal transport rate indicates that the disease has worsened or progressed; an increase in net total transport or net total axonal transport rate indicates that the disease has improved or regressed; and no change in net total transport or net total axonal transport rate indicates that the disease has neither worsened nor improved; wherein determining net total transport and/or net axonal transport rate in the subject comprises locally administering to a muscle of the subject a radiolabeled agent comprising tetanus toxic C fragment; obtaining a first image of the agent in the subject prior to the muscle injection at a first time point; obtaining at least a second image of the agent in the subject at a subsequent time point and then multiple additional images at later time points; determining at each time point after injection the total amount of the agent that is taken up into the motor neurons in the pool that innervates the injected muscle, to determine net total transport at that time point; and optionally using the net total transport at the time points to calculate the rate of uptake, to determine net total axonal transport rate.

In some embodiments, a therapy was administered to the subject between the first and the later time points, and a reduction in net total transport and/or net axonal transport rate indicates that the therapy has had no effect; an increase in net total transport and/or net axonal transport rate indicates that the therapy is effective; and no change indicates that the therapy has stabilized the disease in a progressing subject or neither worsened nor improved the disease in a non-progressing subject. (For example, if the disease is rapidly progressive, no change in these parameters may indicate that a therapy has stabilized the condition). This is particularly useful for evaluating new treatments in clinical trials. In some embodiments, the treatment is riluzole, radicut, Edaravone, inosine, or stem cells.

In some embodiments of the methods described herein, the imaging is performed using computed tomography (CT), magnetic resonance/nuclear magnetic resonance imaging (MRI/NMR), Single photon emission computed tomography (SPECT) or positron emission computed tomography (PET).

In some embodiments of the methods described herein, the methods include using an agent labeled with ⁶⁴Cu, ⁶⁷Ga, ⁸⁶Y, ¹²⁴I, ¹¹¹In, ⁸⁹Zr , or ^{99m}Tc.

In some embodiments of the methods described herein, the subject has or is diagnosed with amyotrophic lateral sclerosis (ALS), peripheral neuropathy, motor neuropathy, or has had a traumatic injury to a nerve.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A. Schematic illustrating the overall structure of Tetanus Toxin, adapted from Calvo, A., et al., Int J Mol Sci. 2012; 13(6):6883-901.

FIG. 1B. Overview of mechanism, highlighting the point that TTC/tetanus toxin is taken up at the distal motor neuron terminal, transported to the cell body, released and subsequently located in the presynaptic terminals of incoming nerve terminals that synapse on the motor neuron. Adapted from Rossetto, O., et al., Toxicon. 2013 May; 66:59-63.

FIG. 2. A set of images illustrating the binding of TTC, and of TTC labeled with cold iodine (exactly as I¹²⁵ is attached to TTC) to NE-RE-105 cells that express the tetanus receptor. The point is that iodination of TTC does not impair binding to the receptors. DAPI staining identifies the cell nuclei; the TTC is recognized using a rabbit polyvalent anti-TTC antibody that, in turn, is recognized using a fluoresceinated anti-rabbit antibody.

FIGS. 3A-C. Sets of images showing that I¹²⁵-TTC injected into the tongue muscle of a rat is taken up in the region of the hypoglossal nucleus in the midbrain. This is viewed in sagittal (FIG. 3A), axial (FIG. 3B) and top (FIG. 3C) views. There is some non-specific uptake that clears, leaving a punctate site of persistent uptake over the hypoglossal nucleus. The topmost image depicts the location of the hypoglossal nucleus in the brainstem.

FIG. 4A. Immunostaining for TTC at the end of the experiment in FIG. 3 documents TTC positivity (within dotted lines, left-most panel) overlying the hypoglossal nucleus, confirming the presence of the protein. The lower figure illustrates the location of the hypoglossal nuclei (from Miana-Mena F J, Roux S, Benichou J-C, Osta R, Brulet. Neuronal activity-dependent membrane traffic at the neuromuscular junction, PNAS 99(5):3234-3239, 200, FIG. 4)

FIG. 4B. Immunostaining for TTC over the anterior horn at the L4 level of the spinal cord of an animal injected ipsilaterally with TTC, as assessed at 12, 24 and 48 hours. This demonstrates a transition from pure intracellular localization at 12 hours, to a mixed localization in both intracellular and extracellular compartments at 24 hours, and predominantly extracellular localization at 48 hours.

FIGS. 5A-B. This shows the time course of the radioactivity as it accumulates in the hypoglossal nuclei in four rats (upper curves, 5A). The pattern of radioactivity in a control region is also illustrated (lower curves, 5A). The averages of these are illustrated in 5B.

FIG. 6. This experiment from 2008 documented uptake of indium-111 labelled TTC from gastrocnemius muscle into the mouse spinal cord as seen at 45 hours in sagittal, coronal and transverse planes (FIG. 6A). The axial views (FIG. 6B) show the appearance of the signal within the spinal cord (see arrows).

FIGS. 7A-B. These figures show the time course of uptake of I¹²⁵-TTC into the mouse spinal cord of the same mouse following injection into the gastrocnemius muscle. The signal is detected at the L4/5 spinal segments as seen in the sagittal and axial planes. This is quite clear by 24 hours; the signal persists through 257 hours. FIG. 8. This shows the comparative uptake of I¹²⁵-TTC in wild-type vs ALS mice (n=6 of each) following gastrocnemius injection. These mice were 85 days old. Data from the six wild-type mice were averaged into a single curve; data from the six ALS mice are also averaged into a single curve. ***: p<0.005

FIG. 9 shows the data from control and ALS mice extended to 340 hours. Data for 45-125 day old control and ALS mice also shown ***: p<0.005

FIG. 10 is a graph showing the uptake of I¹²⁵-labeled tetanus toxoid in two wild-type rats.

FIG. 11 shows that in WT mice aging was associated with a reduction in the peak net axonal transport and a progressive delay in the time to peak. The transport curves (TTC uptake) are shown for WT mice of the following ages: 50, 100, 300, 500 and 700 days. The curves are followed out to approximately 700 hours post injection.

### DETAILED DESCRIPTION

Relatively few tools exist to quantitate the function of motor neurons. These include clinical or quantitative testing of muscle strength; imaging the nerves with methods like computerized tomographic (CT) or magnetic resonance image (MRI) scanning; electrophysiological studies that describe how rapidly and how well the nerves conduct electrical impulses; and performing analyses of biopsies of small motor neurons using microscopic techniques. These methods are helpful in diagnosing motor neuron pathologies, and in distinguishing motor from sensory nerve pathology. However, these methods have limitations: first, they only assess the clinical, morphological and electrical behavior of neurons; it is likely that processes in neurons can malfunction without disturbances of the clinical or electrophysiological properties of neurons; and second, these methods may be relatively insensitive to subtle changes in motor nerve function.

Described herein are new techniques that can assess the status of neurons independently of their clinical or electrophysiological parameters and that provide highly sensitive measurements the status of the motor neuron. These techniques can assist in much earlier diagnosis of motor neuron dysfunction. And, importantly, these methods allow close and sensitive monitoring of the status over time of pathological or at-risk motor neurons. In other words, these methods provide a way to monitor how rapidly a motor neuron deteriorates, or to define subtle improvement in function following treatment. In a disease like amyotrophic lateral sclerosis (ALS, Lou Gehrig's disease) a sensitive biomarker that depicts the state of the motor neuron can greatly accelerate therapy development, as it would be easier and much more rapid to see a therapeutic benefit using a biomarker of axonal function than to use disease survival as an endpoint.

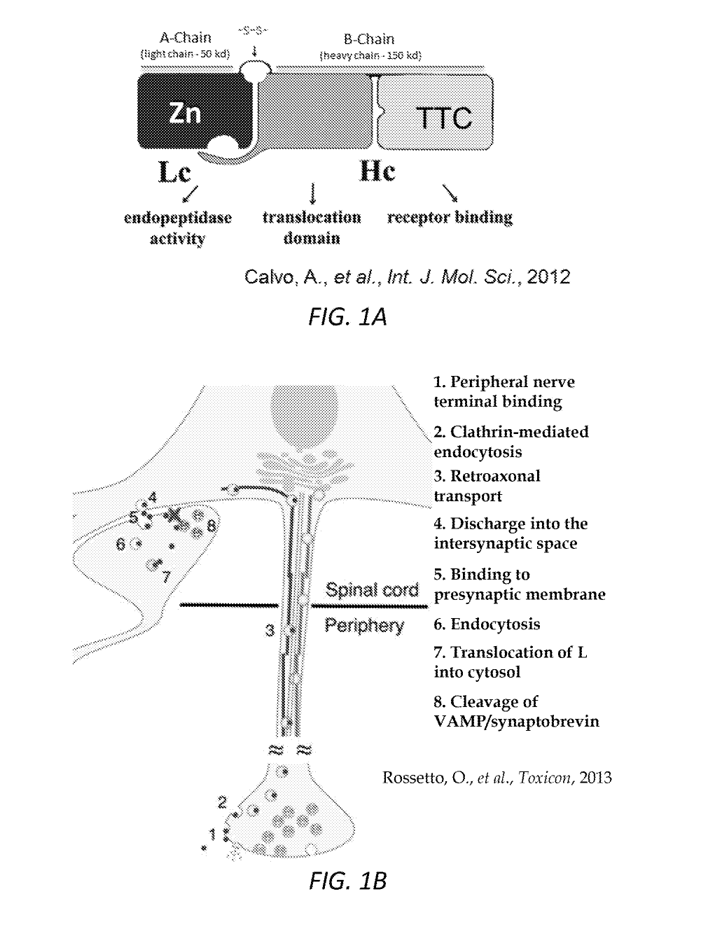

The present methods can be used to quantitate how effectively a motor neuron takes up a marker protein from muscle and carries it retrogradely to the motor neuron cell body in the spinal cord, using the ability of tetanus toxin to be taken up from muscle and carried to the spinal cord. Briefly, tetanus toxin is a highly toxic protein that is produced in by the bacterium clostridia tetani. This bacterium is encountered in wound infections, wherein under anaerobic conditions, this organism produces a neurotoxic protein of 150 kD (tetanus neurotoxin or TeNT). At the site of infection, usually in muscle, this is cleaved into two fragments of 100 kD B-chain (heavy chain) and a 50 kD A- chain (light chain) which are covalently linked via disulfide bonds (See FIG. 1A). The B-chain binds to receptors on the distal motor terminal. The primary receptor is thought to be a surface ganglioside GT1b; other receptors may also be involved including a GPI-linked protein. Entry of the tetanus molecular into the neuron is facilitated by a translocation domain in the B-chain, leading to endocytosis of the toxin via a microlipid domain on the distal neuronal surface. Once incorporated into the distal endosome, the toxin is transported via axonal transport of the endosome from the distal terminal to the motor neuron cell body. At the motor neuron cell body, the endocytic vesicle bearing the tetanus toxin is released to be taken up by interneurons that form synaptic junctions with the motor neuron. After this process of transcytosis to the interneuron, the disulfide bonds in the tetanus toxin within the transcytosed vesicle are reduced. This frees the A-chain, a zinc-endopeptidase, that can then attack key synaptic proteins (SNARE proteins, including synaptobrevin), thereby blocking exocytosis of synaptic vesicles from the interneurons. This disruption of vesicle release impairs both excitatory and inhibitory synaptic inputs. The disruption of inhibitory synaptic inputs mediated by the neurotransmitters glycine and GABA leads to profound electrical hyperactivity of motor neurons (see FIG. 1B). Clinically, this is evident as tetanus, a state in which minimal sensory stimuli produce powerful, painful, debilitating motor spasms.

Importantly, neuronal uptake and retrograde transport of TeNT in the above process depends critically on a 50 kD, carboxy-terminal subcomponent (Hc) of the B-chain (heavy chain). This subcomponent, also designated as tetanus toxin C fragment or TTC, is non-catalytic. This 451 amino acid protein can be safely delivered intramuscularly and shown subsequently to be taken up into motor neurons using methods such as immunostaining of the spinal cord. This property of non-toxic retrograde transport has been documented; TTC can be used to deliver other cargo molecules, such as glial derived neurotrophic factor and survival motor neuron protein to the central nervous system (see, e.g., U.S. Pat. No. 5,780,024). Schellingerhout et al. documented that TTC can be fluorescently labeled and used in a limited manner as a marker for axonal transport in vivo (see LeRoux et al., Mol Imaging Biol. 2014 August; 16(4):504-10; Schellingerhout et al., PLoS One. 2012; 7(9):e45776; Schellingerhout et al., Mol Imaging. 2009 December;8(6):319-29).

Provided herein are non-invasive methods for diagnosing a motor neuron disorder in a living subject, e.g., a subject presenting with one or more symptoms of a neurodegenerative disorder or a subject not presenting a symptom of a neurodegenerative disorder (e.g., an undiagnosed and/or asymptomatic subject). Also provided herein are prognostic methods and methods of monitoring progression of a motor neuron pathology, as well as methods of determining whether a treatment for a motor neuron pathology is having any therapeutic effect, e.g., decreasing the rate of onset or the progression of the disease. The methods use rate of net axonal transport as a measure of disease and/or disease severity. Subjects who can be evaluated according to these methods include mammals, e.g., large mammals including pigs, cows, sheep, horses, cats, dogs, non-human primates and humans. In some embodiments, the subject is an experimental animal; in some embodiments, the subject is a human in a clinical trial.

### Motor Neuron Pathologies

Motor neuron pathologies are a class of neurological pathologies that are characterized by the progressive loss of the structure and function of motor neurons and motor neuronal cell death. Non-limiting examples of motor neuron pathologies include amyotrophic lateral sclerosis, progressive bulbar palsy, pseudobulbar palsy, primary lateral sclerosis, progressive muscular atrophy, spinal muscular atrophy, post-polio syndrome, diverse types of peripheral neuropathy and traumatic nerve injury.

A health care professional may diagnose a subject as having a motor neuron pathology by the assessment of one or more symptoms in the subject. Non-limiting symptoms of a motor neuron pathology in a subject include difficulty lifting the front part of the foot and toes; difficulty lifting the whole leg or standing or walking; weakness in arms, legs, feet, or ankles; hand weakness or clumsiness; muscle cramps and atrophy; twitching in arms, shoulders, torso and legs; stiffness of movement of the arms and legs in some cases; weakness of the tongue and pharyngeal muscles leading to difficulty speaking, chewing and swallowing; and muscle paralysis.

Alternatively, a health care professional may diagnose a subject as having a sensory or a combined sensori-motor neurodegenerative disorder by the assessment of one or more symptoms in the subject such as spontaneous muscle twitching; tingling or pain in parts of body; electric shock sensations that occur with head movements; tremor; unsteady gait; misinterpretation of spatial relationships; loss of automatic movements; impaired posture and balance; stiff muscles; bradykinesia; involuntary jerking or writhing movements (chorea); involuntary, sustained contracture of muscles (dystonia); lack of flexibility; and others known in the art.

A health care professional may also base a diagnosis, in part, on the subject's family history of a motor neuron or neurodegenerative disorder. A health care professional may diagnose a subject as having a motor neuron or neurodegenerative disorder upon presentation of a subject to a health care facility (e.g., a clinic or a hospital). In some instances, a health care professional may diagnose a subject as having a motor neuron disorder while the subject is admitted in an assisted care facility. Typically, a physician diagnoses a motor neuron disorder in a subject after the presentation of one or more symptoms.

### Methods of Imaging Net Axonal Transport

The methods described herein use total amount of uptake and the rate of uptake by cell bodies of motor neurons of an agent injected in the muscle innervated by that pool of motor neurons. The total uptake, also defined as net axonal transport in this study, is a measure of disease and/or disease severity. The rate and extent (amount) of net axonal transport are measured by administering a labeled agent, e.g., TTC or tetanus toxoid, to a subject and imaging the transport of the labeled agent. The methods can include interval imaging, e.g., imaging at intervals of 1, 2, 4, 6, 8, 10, 12, 18, 24, or 48 hours to measure both total quantity of TTC that is taken up and the rate of its uptake into the cell bodies of the motor nerves that innervate the target (injected) muscle. In some embodiments a baseline image is obtained prior to the injection after which subsequent images of the motor neuron cell bodies are obtained at, e.g., 1,2, 6, 12, 18, 24, 36, or 48 hour intervals or more following the injection. Each image can be, but need not be, saved for later analysis; optionally, the image obtained is analyzed without being saved.

The location and quantities of the labeled agent are determined in each of those subsequent images and compared to the baseline image. The key parameters that are determined, as in FIGS. 8, 9 and 10, are the total amount of labeled TTC that is transported (the height of the curve of uptake vs time) and the rate of transport (the time to the peak of the rising phase of the curve). These parameters permit one to calculate the rate and total amount of transport in the subject, which can then be compared to the comparable parameters from a reference (control) subject (e.g., representing a normal subject with no motor neuron pathology) or a disease reference subject, (e.g., representing a subject with a known or categorized severity of motor neuron pathology). Reduction in the total amount of transport or the rate of uptake can indicate either the presence of a pathology in the motor neurons, as might be caused by motor neuron pathology, trauma or neuropathy in the subject (for example, in FIG. 8, the net axonal transport curves for ALS mice are reduced by comparison with the age-matched wildtype mice), the loss of innervation of muscle by the distal motor nerve terminals, or both.

In some embodiments, when the rate and total amount of transport are reduced and subject has one or more symptoms associated with pathology in the motor neuron, such as a motor neuron disease, then the subject is diagnosed with the disease. In some embodiments, the subject has no overt signs or symptoms of motor neuron pathology, but if the rate and total amount of transport are reduced, then the subject has, or has an increased risk of developing motor neuron pathology, such as a motor neuron disease. In some embodiments, once it has been determined that a person has motor neuron pathology, or has an increased risk of developing a motor neuron pathology, then a treatment (such as riluzole, the only FDA-approved therapy for ALS), can be administered.

Suitable reference values can be determined using methods known in the art, e.g., using standard clinical trial methodology and statistical analysis. The reference values can have any relevant form. In some cases, the reference comprises a predetermined value for a meaningful rate and quantity of net axonal transport, e.g., a control reference that represents a normal amount and/or rate of transport, as might be defined in an unaffected subject or a subject who is not at risk of developing a disease described herein, and/or a disease reference that represents a rate and quantity of net axonal transport associated with motor neuron pathology.

The predetermined total amount of transport or and/or rate of transport can be a single cut-off (threshold) value, such as a median or mean values that define the boundaries of an upper or lower quartile, tertile, or other segment of a clinical trial population that is determined to be statistically different from the same fractional segment in a control population. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, a range of reference distances traveled and/or rates of transport are used, e.g., to determine how severe a disease is in a subject. For example, a subject who has a total amount of transport or and/or rate of transport that falls within a given range may be identified as having severe disease, moderate disease, or mild disease, depending on which range the distance traveled and/or rate of transport fall within.

Subjects associated with predetermined values are typically referred to as reference subjects. For example, in some embodiments, a control reference subject does not have a disorder that entails motor pathology, such as motor neuron pathology.

A disease reference subject is one who has (or has an increased risk of developing) pathology of the motor neurons, such as motor neuron pathology or neuropathy. An increased risk is defined as a risk above the risk of subjects in the general population.

Thus, in some cases the total amount of transport or and/or rate of transport in a subject being reduced relative to a reference total transport or rate of transport is indicative of a clinical status (e.g., indicative of a motor neuron pathology). In other cases total amount of transport or and/or rate of transport in a subject being greater than/faster or equal to the reference distance traveled and/or rate of transport is indicative of the absence of disease or normal risk of motor neuron pathology. In some embodiments, the amount by which total amount of transport or and/or rate of transport in the subject is reduced relative to control parameters is sufficient to distinguish a subject from a control subject, and optionally is a statistically significantly less/slower than the total amount of transport or and/or rate of transport in a control subject. In cases where total amount of transport or and/or rate of transport in a subject are equal to the reference total amount of transport or and/or rate of transport, the “being equal” refers to being approximately equal (e.g., not statistically different).

The predetermined value can depend upon the particular population of subjects (e.g., human subjects) selected. For example, an apparently healthy population will have a different ‘normal’ range of distance traveled and/or rate of transport than will a population of subjects which have, are likely to have, or are at greater risk to have pathology in the motor neurons, such as a motor neuron disorder described herein. Accordingly, the predetermined values selected for distance traveled and/or rate of transport may take into account the category (e.g., sex, age, height, weight, health, risk, presence of other pathologies) in which a subject (e.g., human subject) falls. Appropriate ranges and categories can be selected with no more than routine experimentation by those of ordinary skill in the art.

In characterizing likelihood, or risk, numerous predetermined values can be established.

In some embodiments, the predetermined total amount of transport or and/or rate of transport at a first time point can be compared to total amount of transport or and/or rate of transport at a later time point. A decrease in these parameters can indicate that the disease has worsened or progressed, whereas an increase in distance traveled and/or faster rate of transport can indicate that the disease has improved or regressed, and no change in distance traveled and/or rate of transport indicates that the disease has neither worsened nor improved. In the context of a therapy, the methods can be used to determine efficacy of a therapy administered in the intervening time between the first and the later time points, such that a decrease total amount of transport or and/or rate of transport can indicate that the therapy has had no effect, whereas an increase these parameters can indicate that the therapy is effective; no change total amount of transport or and/or rate of transport indicates that the therapy has neither worsened nor improved the disease in the subject. Thus, these methods can be used in subjects to monitor therapy, and to determine quickly whether a treatment, e.g., an experimental treatment in a clinical trial, is effective in the subject. For example, these methods can be used to determine whether a treatment for ALS is effective in a subject. At present, valid testing of a drug for ALS typically requires upwards of a year or more, because there is no sensitive, rapid measure to determine whether the compound in question is having an immediately beneficial impact on motor neurons. One must follow a large cohort of patients to look for a beneficial clinical impact (such as improved strength or survival). The present methods provide an alternative, a surrogate marker that can indicate improvement in a critical parameter over a short time frame and therefore predict the outcome of a full, longer clinical trial. These methods accelerate the pace of a clinical trial while reducing the number of required subjects, resulting in greater efficiencies, cost savings and, above all, a faster pace toward therapy discovery.

The methods described herein can use any imaging modality suitable for imaging the labeled agents in living subjects. Suitable imaging methods include nuclear imaging method such as computed tomography (CT), magnetic resonance/nuclear magnetic resonance imaging (MRI/NMR), Single photon emission computed tomography (SPECT) or positron emission computed tomography (PET), using an agent labeled, e.g., with ⁶⁴Cu, ⁶⁷Ga, ⁸⁶Y, ¹²⁴I, ¹¹¹In, ⁸⁹Zr, or ^{99m}Tc. See, e.g., Den et al., Nucl Med Biol. 2013 January; 40(1): 3-14.

In the present methods, the labeled agents include the fragment TTC or the full TET protein. Exemplary sequences follow:

| Synthetic construct gene for Hc fragment of tetanus toxin | |
|---|---|
| (GenBank: AM412776.1) DNA Sequence: 1419 bp | |
| (SEQ ID NO: 1) | |
| 0001 | atgggcagca gccatcatca tcatcatcac agcagcggcc tggtgccgcg cggcagccat |
| 0061 | atgaaaaacc ttgattgttg ggtcgacaac gaagaagaca tcgatgttat cctgaaaaag |
| 0121 | tctaccattc tgaacttgga catcaacaac gatattatct ccgacatctc tggtttcaac |
| 0181 | tcctctgtta tcacatatcc agatgctcaa ttggtgccgg gcatcaacgg caaagctatc |
| 0241 | cacctggtta acaacgaatc ttctgaagtt atcgtgcaca aggccatgga catcgaatac |
| 0301 | aacgacatgt tcaacaactt caccgttagc ttctggctgc gcgttccgaa agtttctgct |
| 0361 | tcccacctgg aacagtacgg cactaacgag tactccatca tcagctctat gaagaaacac |
| 0421 | tccctgtcca tcggctctgg ttggtctgtt tccctgaagg gtaacaacct gatctggact |
| 0481 | ctgaaagact ccgcgggcga agttcgtcag atcactttcc gcgacctgcc ggacaagttc |
| 0541 | aacgcgtacc tggctaacaa atgggttttc atcactatca ctaacgatcg tctgtcttct |
| 0601 | gctaacctgt acatcaacgg cgttctgatg ggctccgctg aaatcactgg tctgggcgct |
| 0661 | atccgtgagg acaacaacat cactcttaag ctggaccgtt gcaacaacaa caaccagtac |
| 0721 | gtatccatcg acaagttccg tatcttctgc aaagcactga acccgaaaga gatcgaaaaa |
| 0781 | ctgtatacca gctacctgtc tatcaccttc ctgcgtgact tctggggtaa cccgctgcgt |
| 0841 | tacgacaccg aatattacct gatcccggta gcttctagct ctaaagacgt tcagctgaaa |
| 0901 | aacatcactg actacatgta cctgaccaac gcgccgtcct acactaacgg taaactgaac |
| 0961 | atctactacc gacgtctgta caacggcctg aaattcatca tcaaacgcta cactccgaac |
| 1021 | aacgaaatcg attctttcgt taaatctggt gacttcatca aactgtacgt ttcttacaac |
| 1081 | aacaacgaac acatcgttgg ttacccgaaa gacggtaacg ctttcaacaa cctggacaga |
| 1141 | attctgcgtg ttggttacaa cgctccgggt atcccgctgt acaaaaaaat ggaagctgtt |
| 1201 | aaactgcgtg acctgaaaac ctactctgtt cagctgaaac tgtacgacga caaaaacgct |
| 1261 | tctctgggtc tggttggtac ccacaacggt cagatcggta acgacccgaa ccgtgacatc |
| 1321 | ctgatcgctt ctaactggta cttcaaccac ctgaaagaca aaatcctggg ttgcgactgg |
| 1381 | tacttcgttc cgaccgatga aggttggacc aacgactaa |
| Hc fragment of tetanus toxin, Protein Sequence: 472 aa | |
| (SEQ ID NO: 2) | |
| 001 | MGSSHHHHHH SSGLVPRGSH MKNLDCWVDN EEDIDVILKK STILNLDINN DIISDISGFN |
| 061 | SSVITYPDAQ LVPGINGKAI HLVNNESSEV IVHKAMDIEY NDMFNNFTVS FWLRVPKVSA |
| 121 | SHLEQYGTNE YSIISSMKKH SLSIGSGWSV SLKGNNLIWT LKDSAGEVRQ ITFRDLPDKF |
| 181 | NAYLANKWVF ITITNDRLSS ANLYINGVLM GSAEITGLGA IREDNNITLK LDRCNNNNQY |
| 241 | VSIDKFRIFC KALNPKEIEK LYTSYLSITF LRDFWGNPLR YDTEYYLIPV ASSSKDVQLK |
| 301 | NITDYMYLTN APSYTNGKLN IYYRRLYNGL KFIIKRYTPN NEIDSFVKSG DFIKLYVSYN |
| 361 | NNEHIVGYPK DGNAFNNLDR ILRVGYNAPG IPLYKKMEAV KLRDLKTYSV QLKLYDDKNA |
| 421 | SLGLVGTHNG QIGNDPNRDI LIASNWYFNH LKDKILGCDW YFVPTDEGWT ND |
| Clostridium tetani gene for tetanus toxin (GenBank: X06214.1), | |
| DNA Sequence: 4338 bp | |
| (SEQ ID NO: 3) | |
| 0001 | gcttcatatg agtcgtcaag ctgtatataa aaataaggtt ttagcattaa aaaaattaga |
| 0061 | acctatagta aataaattaa ttaatatata gtttttataa tttaattatg aataatattc |
| 0121 | ttaagataaa aagtaaattt ttaaaaattt aaattttcag tttacaaaaa ataacctgat |
| 0181 | tatgttatat gtaattgtaa aaaacatata aaaaatcaga aaaatttagg aggtatatta |
| 0241 | ttaatggatt aaataataat tttttaattt acttttgatt aataaatatt aaatgtttat |
| 0301 | tttaattagg agatgatacg tatgccaata accataaata attttagata tagtgatcct |
| 0361 | gttaataatg atacaattat tatgatggag ccaccatact gtaagggtct agatatctat |
| 0421 | tataaggctt tcaaaataac agatcgtatt tggatagtgc cggaaaggta tgaatttggg |
| 0481 | acaaaacctg aagattttaa cccaccatct tcattaatag aaggtgcatc tgagtattac |
| 0541 | gatccaaatt atttaaggac tgattctgat aaagatagat ttttacaaac catggtaaaa |
| 0601 | ctgtttaaca gaattaaaaa caatgtagca ggtgaagcct tattagataa gataataaat |
| 0661 | gccatacctt accttggaaa ttcatattcc ttactagaca agtttgatac aaactctaat |
| 0721 | tcagtatctt ttaatttatt agaacaagac cccagtggag caactacaaa atcagcaatg |
| 0781 | ctgacaaatt taataatatt tggacctggg cctgttttaa ataaaaatga ggttagaggt |
| 0841 | attgtattga gggtagataa taaaaattac ttcccatgta gagatggttt tggctcaata |
| 0901 | atgcaaatgg cattttgccc agaatatgta cctacctttg ataatgtaat agaaaatatt |
| 0961 | acgtcactca ctattggcaa aagcaaatat tttcaagatc cagcattact attaatgcac |
| 1021 | gaacttatac atgtactaca tggtttatac ggaatgcagg tatcaagcca tgaaattatt |
| 1081 | ccatccaaac aagaaattta tatgcagcat acatatccaa taagtgctga agaactattc |
| 1141 | acttttggcg gacaggatgc taatcttata agtattgata taaaaaacga tttatatgaa |
| 1201 | aaaactttaa atgattataa agctatagct aacaaactta gtcaagtcac tagctgcaat |
| 1261 | gatcccaaca ttgatattga tagctacaaa caaatatatc aacaaaaata tcaattcgat |
| 1321 | aaagatagca atggacaata tattgtaaat gaggataaat ttcagatact atataatagc |
| 1381 | ataatgtatg gttttacaga gattgaattg ggaaaaaaat ttaatataaa aactagactt |
| 1441 | tcttatttta gtatgaatca tgaccctgta aaaattccaa atttattaga tgatacaatt |
| 1501 | tacaatgata cagaaggatt taatatagaa agcaaagatc tgaaatctga atataaagga |
| 1561 | caaaatatga gggtaaatac aaatgctttt agaaatgttg atggatcagg cctagtttca |
| 1621 | aaacttattg gcttatgtaa aaaaattata ccaccaacaa atataagaga aaatttatat |
| 1681 | aatagaactg catcattaac agatttagga ggagaattat gtataaaaat taaaaatgaa |
| 1741 | gatttaactt ttatagctga aaaaaatagc ttttcagaag aaccatttca agatgaaata |
| 1801 | gttagttata atacaaaaaa taaaccatta aattttaatt attcgctaga taaaattatt |
| 1861 | gtagattata atctacaaag taaaattaca ttacctaatg ataggacaac cccagttaca |
| 1921 | aaaggaattc catatgctcc agaatataaa agtaatgctg caagtacaat agaaatacat |
| 1981 | aatattgatg acaatacaat atatcaatat ttgtatgctc aaaaatctcc tacaactcta |
| 2041 | caaagaataa ctatgactaa ttctgttgat gacgcattaa taaattccac caaaatatat |
| 2101 | tcatattttc catctgtaat cagtaaagtt aaccaaggtg cacaaggaat tttattctta |
| 2161 | cagtgggtga gagatataat tgatgatttt accaatgaat cttcacaaaa aactactatt |
| 2221 | gataaaattt cagatgtatc cactattgtt ccttatatag gacccgcatt aaacattgta |
| 2281 | aaacaaggct atgagggaaa ctttataggc gctttagaaa ctaccggagt ggttttatta |
| 2341 | ttagaatata ttccagaaat tactttacca gtaattgcag ctttatctat agcagaaagt |
| 2401 | agcacacaaa aagaaaagat aataaaaaca atagataact ttttagaaaa aagatatgaa |
| 2461 | aaatggattg aagtatataa actagtaaaa gcaaaatggt taggcacagt taatacgcaa |
| 2521 | ttccaaaaaa gaagttatca aatgtataga tctttagaat atcaagtaga tgcaataaaa |
| 2581 | aaaataatag actatgaata taaaatatat tcaggacctg ataaggaaca aattgccgac |
| 2641 | gaaattaata atctgaaaaa caaacttgaa gaaaaggcta ataaagcaat gataaacata |
| 2701 | aatatattta tgagggaaag ttctagatca tttttagtta atcaaatgat taacgaagct |
| 2761 | aaaaagcagt tattagagtt tgatactcaa agcaaaaata ttttaatgca gtatataaaa |
| 2821 | gcaaattcta aatttatagg tataactgaa ctaaaaaaat tagaatcaaa aataaacaaa |
| 2881 | gttttttcaa caccaattcc attttcttat tctaaaaatc tggattgttg ggttgataat |
| 2941 | gaagaagata tagatgttat attaaaaaag agtacaattt taaatttaga tattaataat |
| 3001 | gatattatat cagatatatc tgggtttaat tcatctgtaa taacatatcc agatgctcaa |
| 3061 | ttggtgcccg gaataaatgg caaagcaata catttagtaa acaatgaatc ttctgaagtt |
| 3121 | atagtgcata aagctatgga tattgaatat aatgatatgt ttaataattt taccgttagc |
| 3181 | ttttggttga gggttcctaa agtatctgct agtcatttag aacaatatgg cacaaatgag |
| 3241 | tattcaataa ttagctctat gaaaaaacat agtctatcaa taggatctgg ttggagtgta |
| 3301 | tcacttaaag gtaataactt aatatggact ttaaaagatt ccgcgggaga agttagacaa |
| 3361 | ataactttta gggatttacc tgataaattt aatgcttatt tagcaaataa atgggttttt |
| 3421 | ataactatta ctaatgatag attatcttct gctaatttgt atataaatgg agtacttatg |
| 3481 | ggaagtgcag aaattactgg tttaggagct attagagagg ataataatat aacattaaaa |
| 3541 | ctagatagat gtaataataa taatcaatac gtttctattg ataaatttag gatattttgc |
| 3601 | aaagcattaa atccaaaaga gattgaaaaa ttatacacaa gttatttatc tataaccttt |
| 3661 | ttaagagact tctggggaaa ccctttacga tatgatacag aatattattt aataccagta |
| 3721 | gcttctagtt ctaaagatgt tcaattgaaa aatataacag attatatgta tttgacaaat |
| 3781 | gcgccatcgt atactaacgg aaaattgaat atatattata gaaggttata taatggacta |
| 3841 | aaatttatta taaaaagata tacacctaat aatgaaatag attcttttgt taaatcaggt |
| 3901 | gattttatta aattatatgt atcatataac aataatgagc acattgtagg ttatccgaaa |
| 3961 | gatggaaatg cctttaataa tcttgataga attctaagag taggttataa tgccccaggt |
| 4021 | atccctcttt ataaaaaaat ggaagcagta aaattgcgtg atttaaaaac ctattctgta |
| 4081 | caacttaaat tatatgatga taaaaatgca tctttaggac tagtaggtac ccataatggt |
| 4141 | caaataggca acgatccaaa tagggatata ttaattgcaa gcaactggta ctttaatcat |
| 4201 | ttaaaagata aaattttagg atgtgattgg tactttgtac ctacagatga aggatggaca |
| 4261 | aatgattaaa cagattgata tgttcatgat tactctatat aaaaaattaa ataatataac |
| 4321 | aatctagcta tattattt |
| Tetanus toxin (GenBank: KIG19893.1), Protein Sequence: 1315 aa | |
| (SEQ ID NO: 4) | |
| 0001 | MPITINNFRY SDPVNNDTII MMEPPYCKGL DIYYKAFKIT DRIWIVPERY EFGTKPEDFN |
| 0061 | PPSSLIEGAS EYYDPNYLRT DSDKDRFLQT MVKLFNRIKN NVAGEALLDK IINAIPYLGN |
| 0121 | SYSLLDKFDT NSNSVSFNLL EQDPSGATTK SAMLTNLIIF GPGPVLNKNE VRGIVLRVDN |
| 0181 | KNYFPCRDGF GSIMQMAFCP EYVPTFDNVI ENITSLTIGK SKYFQDPALL LMHELIHVLH |
| 0241 | GLYGMQVSSH EIIPSKQEIY MQHTYPISAE ELFTFGGQDA NLISIDIKND LYEKTLNDYK |
| 0301 | AIANKLSQVT SCNDPNIDID SYKQIYQQKY QFDKDSNGQY IVNEDKFQIL YNSIMYGFTE |
| 0361 | IELGKKFNIK TRLSYFSMNH DPVKIPNLLD DTIYNDTEGF NIESKDLKSE YKGQNMRVNT |
| 0421 | NAFRNVDGSG LVSKLIGLCK KIIPPTNIRE NLYNRTASLT DLGGELCIKI KNEDLTFIAE |
| 0481 | KNSFSEEPFQ DEIVSYNTKN KPLNFNYSLD KIIVDYNLQS KITLPNDRTT PVTKGIPYAP |
| 0541 | EYKSNAASTI EIHNIDDNTI YQYLYAQKSP TTLQRITMTN SVDDALINST KIYSYFPSVI |
| 0601 | SKVNQGAQGI LFLQWVRDII DDFTNESSQK TTIDKISDVS TIVPYIGPAL NIVKQGYEGN |
| 0661 | FIGALETTGV VLLLEYIPEI TLPVIAALSI AESSTQKEKI IKTIDNFLEK RYEKWIEVYK |
| 0721 | LVKAKWLGTV NTQFQKRSYQ MYRSLEYQVD AIKKIIDYEY KIYSGPDKEQ IADEINNLKN |
| 0781 | KLEEKANKAM ININIFMRES SRSFLVNQMI NEAKKQLLEF DTQSKNILMQ YIKANSKFIG |
| 0841 | ITELKKLESK INKVFSTPIP FSYSKNLDCW VDNEEDIDVI LKKSTILNLD INNDIISDIS |
| 0901 | GFNSSVITYP DAQLVPGING KAIHLVNNES SEVIVHKAMD IEYNDMFNNF TVSFWLRVPK |
| 0961 | VSASHLEQYG TNEYSIISSM KKHSLSIGSG WSVSLKGNNL IWTLKDSAGE VRQITFRDLP |
| 1021 | DKFNAYLANK WVFITITNDR LSSANLYING VLMGSAEITG LGAIREDNNI TLKLDRCNNN |
| 1081 | NQYVSIDKFR IFCKALNPKE IEKLYTSYLS ITFLRDFWGN PLRYDTEYYL IPVASSSKDV |
| 1141 | QLKNITDYMY LTNAPSYTNG KLNIYYRRLY NGLKFIIKRY TPNNEIDSFV KSGDFIKLYV |
| 1201 | SYNNNEHIVG YPKDGNAFNN LDRILRVGYN APGIPLYKKM EAVKLRDLKT YSVQLKLYDD |
| 1261 | KNASLGLVGT HNGQIGNDPN RDILIASNWY FNHLKDKILG CDWYFVPTDE GWTND |

In some embodiments, an agent for use in a method described herein includes an amino acid sequence that is at least about 80% or more identical to the entire length of the sequence shown in SEQ ID NO:2, and retains substantially the same ability to bind to the tetanus receptor, e.g., to NE-RE-105 cells that express the tetanus receptor as described herein. In some embodiments, the nucleotide sequence is at least about 90%, 91%, 92%, 93%, 94%, 95%, 96%, 97%, 98%, 99% or 100% identical to SEQ ID NO:2. The agents can be produced using methods known in the art, e.g., using standard protein production (e.g., by recombinant expression in vitro) and purification methods, and labeled using known chemistries, e.g., as described herein or known in the art.

### Pharmaceutical Compositions and Methods of Administration

The methods described herein include the use of pharmaceutical compositions comprising a labeled agent comprising TTC or TeNT.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Pharmaceutical compositions are typically formulated to be compatible with its intended route of administration, the present methods will typically include local intramuscular injection thus formulation for parenteral administration is desirable.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

The pharmaceutical compositions can be included in a container, pack, or dispenser together with instructions for administration.

### EXAMPLES

The invention is further described in the following examples, which do not limit the scope of the invention described in the claims.

### Materials and Methods

The following materials and methods were used in the Examples below.

Recombinant Expression, Purification, and Radiolabeling of TTC. Recombinant TTC (tetanus toxin residues 865-1315) was overexpressed using plasmid pET28a in Escherichia coli host BL21(DE3) (Novagen). The optimization of TTC expression in host BL21 was achieved and described previously by subcloning a BamHI/HindIII fragment containing a codon-substituted TTC cDNA (Makoff et al., 1989) from plasmid pMALTetC (Figueirdeo et al., 1995) to pET28a. The recombinant TTC proteins were extracted from bacterial culture and purified by immobilized metal affinity chromatography (IMAC) followed by ion exchange chromatography. For IMAC, Histidine-tagged TTC was bound to nickel NTA agarose (Qiagen) and eluted by 200 mM of imidazole. For SP Sepharose cation exchange (GE Healthcare), IMAC-purified samples were first dialyzed against 20 mM of 2-[N-morpholino]ethanesulfonic acid (MES) buffer at a pH of 5.8 and were chromatographed with SP Sepharose resin. The bound material was eluted with 600 mM of NaCl. For Q Sepharose anion exchange chromatography (Amersham Biosciences), the SP-column-purified samples were dialyzed against 20 mM of BisTris buffer at a pH of 6.5 and were chromatographed with Q Sepharse resin. The flowthrough was first dialyzed against 20 mM PBS at a pH of 7.5 and was concentrated using a Centricon Plus-20 centrifugal filter unit (Millipore). The recombinant TTC was purified using a Sephacryl S-300 column (GE Healthcare).

For iodination of TTC, Na¹²⁵I (Perkin Elmer) with a total radioactivity of 10 mCi was added to 0.3 mg of TTC or tetanus toxoid. The iodinated samples were incubated for 10 minutes before quenching the reaction by addition of tyrosine. The labeling efficiency of TTC and tetanus toxoid was determined by instant thin layer chromatography (Agilent).

Binding of TTC in N18-RE-105 Cells. N18-RE-105 neuroblastoma cells were cultured in DMEM (Gibco) and supplemented with HT supplement (Gibco), 400 mM aminopterin (Sigma Aldrich), 10% fetal calf serum (Gibco), and 1% penicillin/streptomycin (Gibco) and passaged at subconfluence. To assess binding and internalization of the radiolabeled-TTC, N18-RE-105 cells (10⁶ cells/mL) were incubated with iodinated-TTC (final concentration 2.5 μg/mL) for 1 hour at 37° C. The cells were fixed with 4% paraformaldehyde (PFA) in PBS for 10 min followed by extensive wash with PBS. The cells were then incubated with rabbit polyclonal antibody against TTC (dilution 1:500; Rockland) and then with a FITC-conjugated fluorescent anti-rabbit secondary antibody (dilution 1:1000; Abcam). Cells were mounted in ProLong Diamond antifade mountant (Life Technologies).

In Vivo Injections of TTC and Tetanus Toxoid. Eight-week-old Sprague Dawley rats were obtained from Taconic, and eight-week-old transgenic mice carrying a human SOD1 mutant (B6SJL-Tg[SOD1-G93A]1Gur/J) were obtained from Jackson Laboratories. To block thyroid uptake of free ¹²⁵I, potassium iodine was added to the drinking water of the animals at least a week before the injection. For tongue muscle injection, a Hamilton syringe (20 μl per animal) was used to inject 5 μg/μl of radiolabeled TTC or tetanus toxoid into the left side of the tongue muscle while the animals were under anesthesia with isoflurane. For leg muscle injection, 20 μl of 5 μg/μl of radiolabeled TTC or tetanus toxoid was injected into the gastrocnemius muscles of the right leg of the anesthetized animal. For animals who underwent sciatic nerve lesion procedure, an incision was made to expose the right sciatic nerve of the anesthetized animals. After the nerve was cut, the incision was sutured closed; these animals were injected with the radiolabeled reagents at post op day 3. Animals were kept alive for seven to fourteen days to allow transportation of the injected proteins. At the endpoint of the experiments, animals were sacrificed by intracardiac perfusion with 4% PFA, followed by harvesting the brains and spinal cords for immunohistochemistry. These harvested tissues were fixed in 4% PFA in PBS for 24 hours and were transferred in 20% sucrose in PBA for 24 hours. The tissues were then frozen in Tissue-Tek O.C.T medium (Sakura) at −80° C. until cryostat sectioning. All animal protocols were approved by the IACUC committee at the University of Massachusetts Medical School.

Immunohistochemistry. For immunohistochemistry, the brain and spinal cord tissues were sectioned using a cryostat with a section thickness of 30 μm. The tissue sections were incubated in PBS with 0.4% Triton X-100, 4% donkey serum, and 4% bovine serum albumin (Sigma Aldrich) for 3 hours, followed by incubation with rabbit polyclonal antibody against TTC (dilution 1:500; Rockland). After extensive wash with PBS, the sections were incubated with a FITC-conjugated fluorescent anti-rabbit secondary antibody (dilution 1:1000; Abcam). The sections were mounted in ProLong Diamond antifade mountant (Life Technologies).

SPECT/CT Imaging. Animals were imaged on a NanoSPECT/CT™ small animal imaging camera (Bioscan Inc.) at various time points following injection. CT scanning was performed at standard resolution, using a 45 kVp voltage and 500 milliseconds exposure time. The SPECT image parameters were 1.0 mm/pixel, 256×256 frame size and 60 sec per projection with 24 projections. During imaging, the animals were anesthetized with 1-2% isoflurane in 1.5 L/min oxygen. The CT and SPECT reconstructions and volume-of-interest (VOI) analysis of the SPECT acquisitions were performed by VivoQuant 1.23 software (InvivoCRO).

### Example 1. Net Axonal Transport of Radiolabeled TTC and Tetanus Toxoid

The present data demonstrates that we can prepare and label TTC using radioactive ligands to quantify the amounts of this protein taken up by retrograde axonal transport in rat and mouse motor neurons, as studied serially in the same animal.

TTC is synthesized and purified using recombinant methods as described above. The imaging studies in these experiments used SPECT imaging; SPECT (single photon emission computed tomography) is a form of nuclear isotopic imaging that allows one to reconstruct the 3-D location of the labeled proteins. Using relatively long half-life isotopes (primarily iodine-¹²⁵ but also indium-¹¹¹) we made the following observations relevant to this patent application.

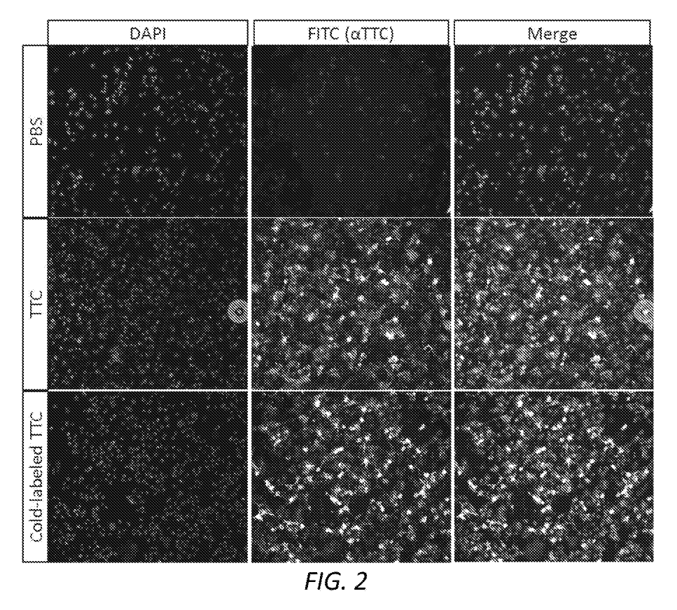

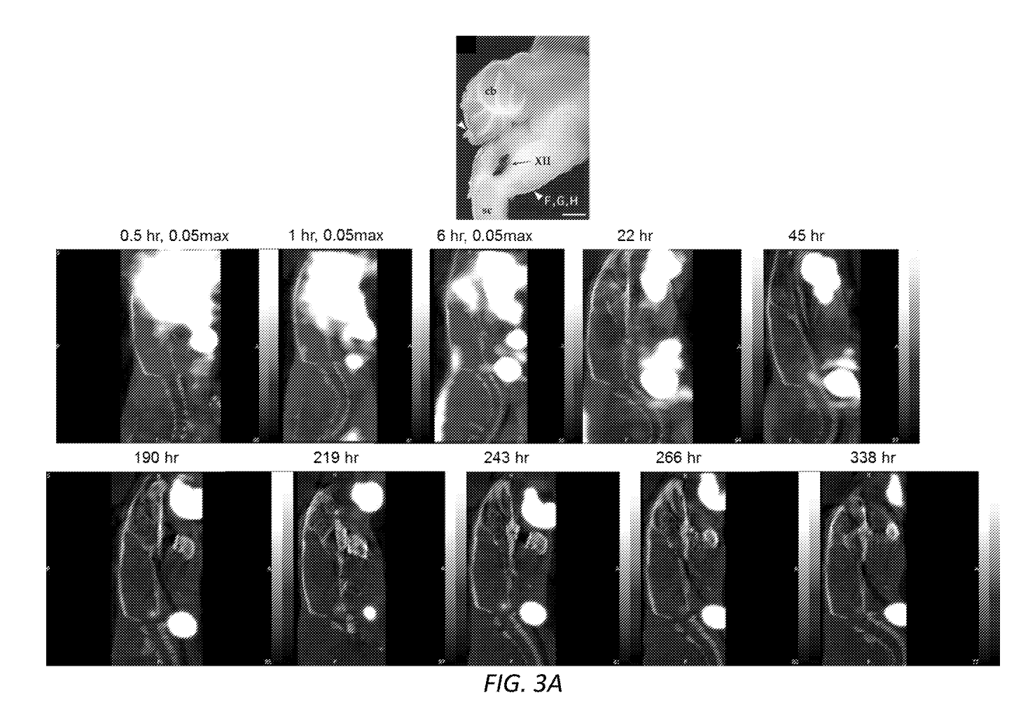

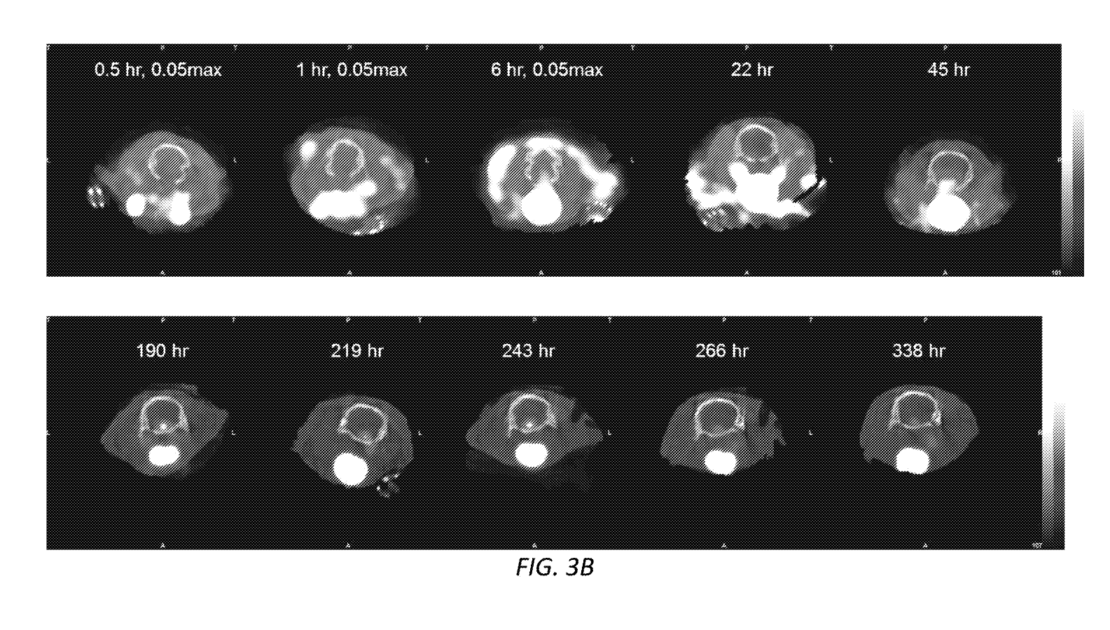

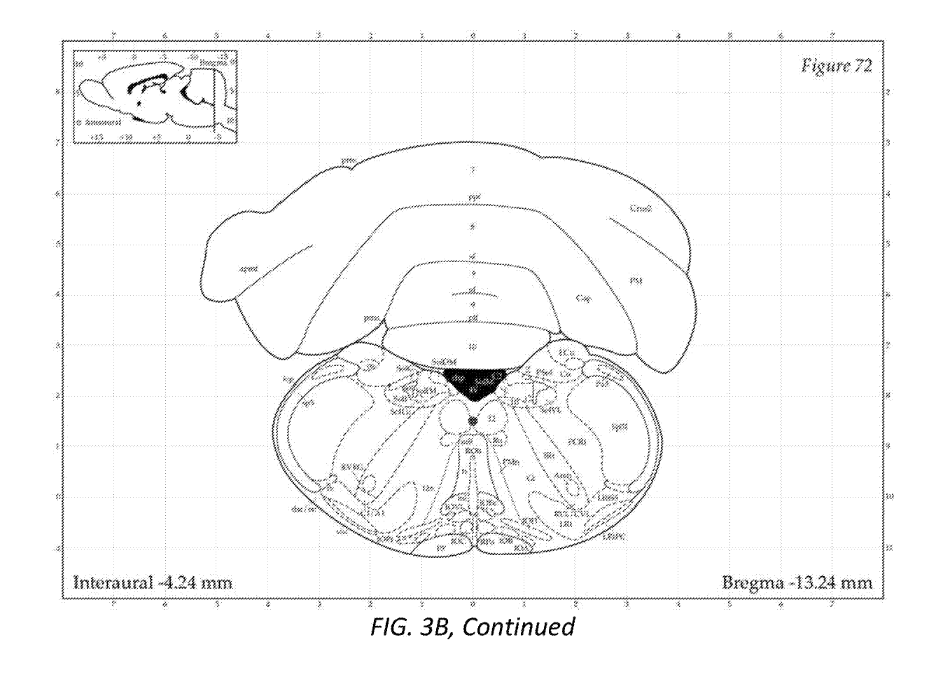

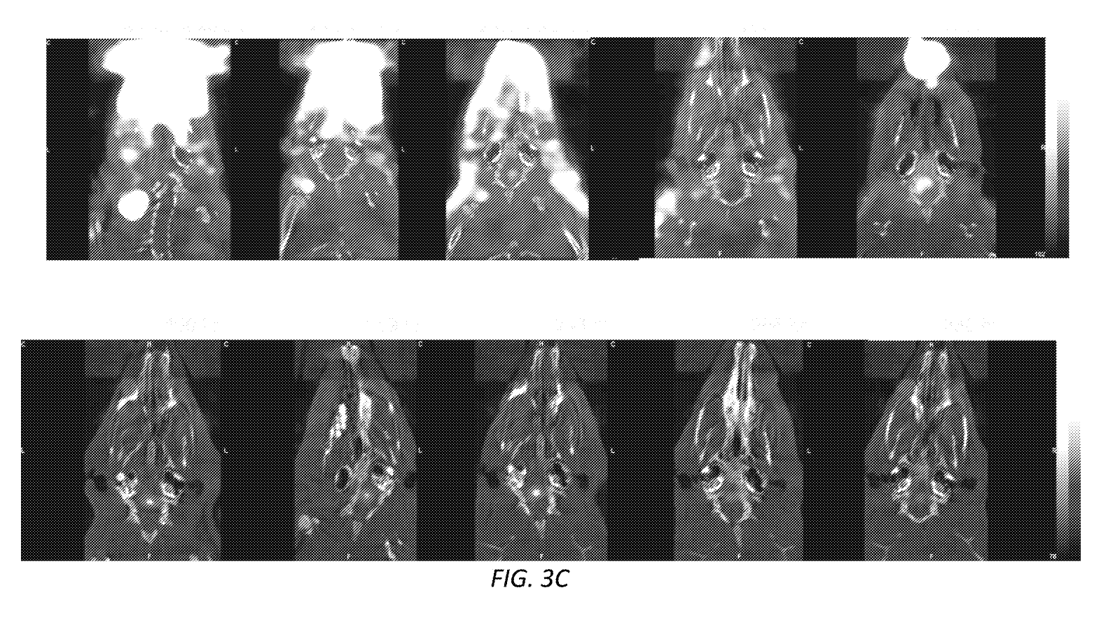

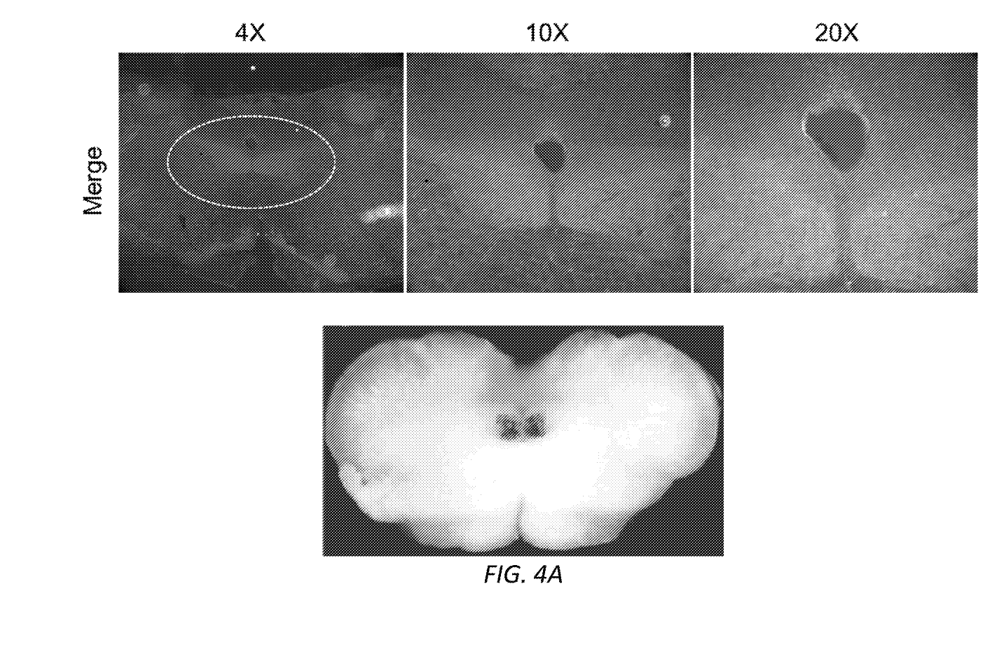

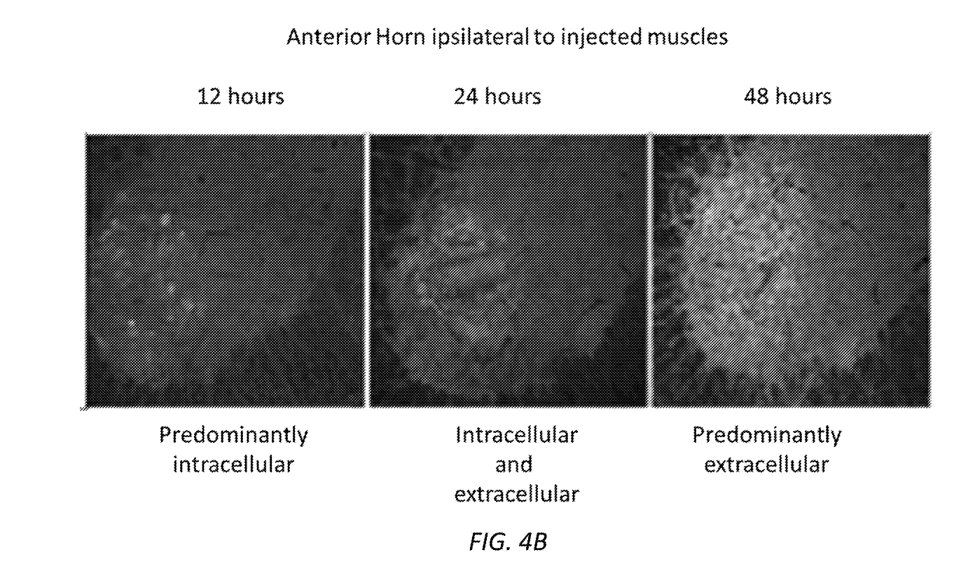

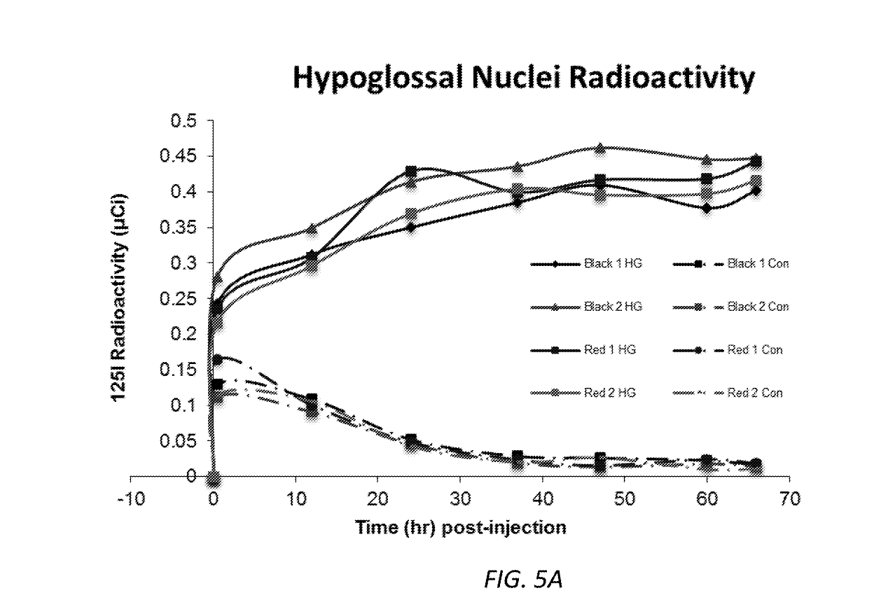

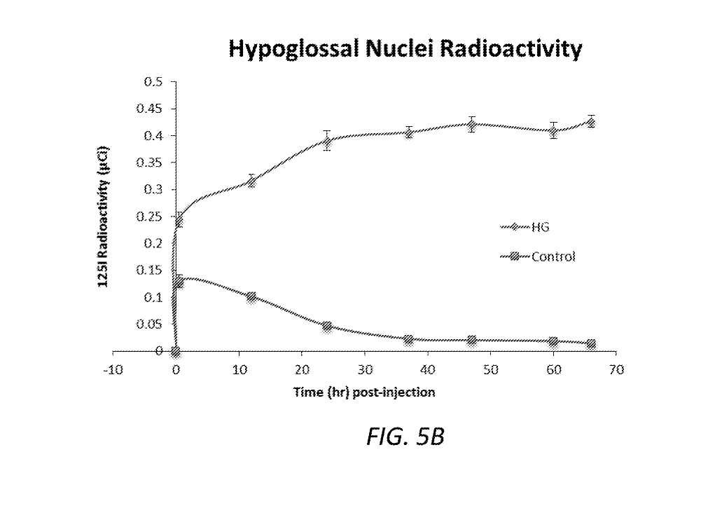

TTC-I¹²⁵ retained the ability to bind to tetanus receptors in cells in culture (FIG. 2). In normal, wildtype rats, when TTC-I¹²⁵ was injected into the tongue, it was shown to be transported to hypoglossal motor neurons, with a characteristic time course for delivery (FIG. 3, 4, 5).

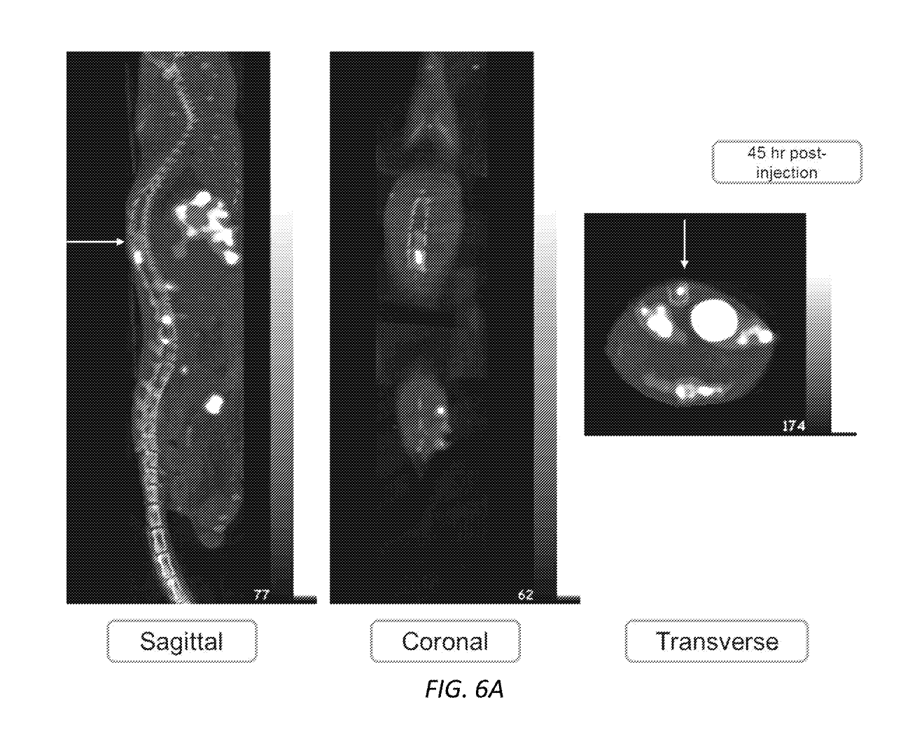

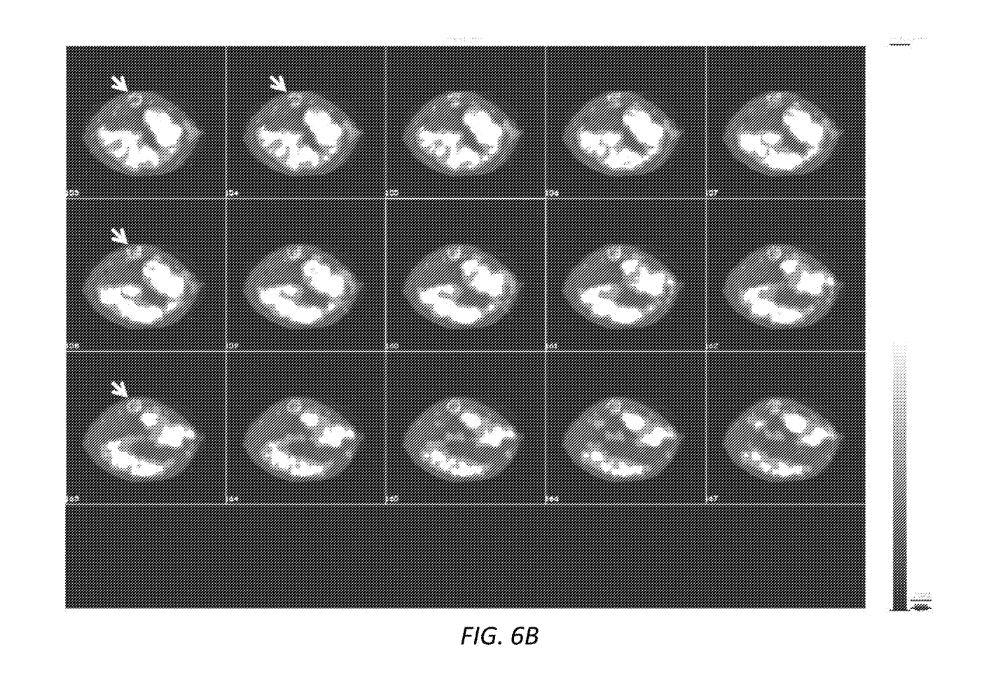

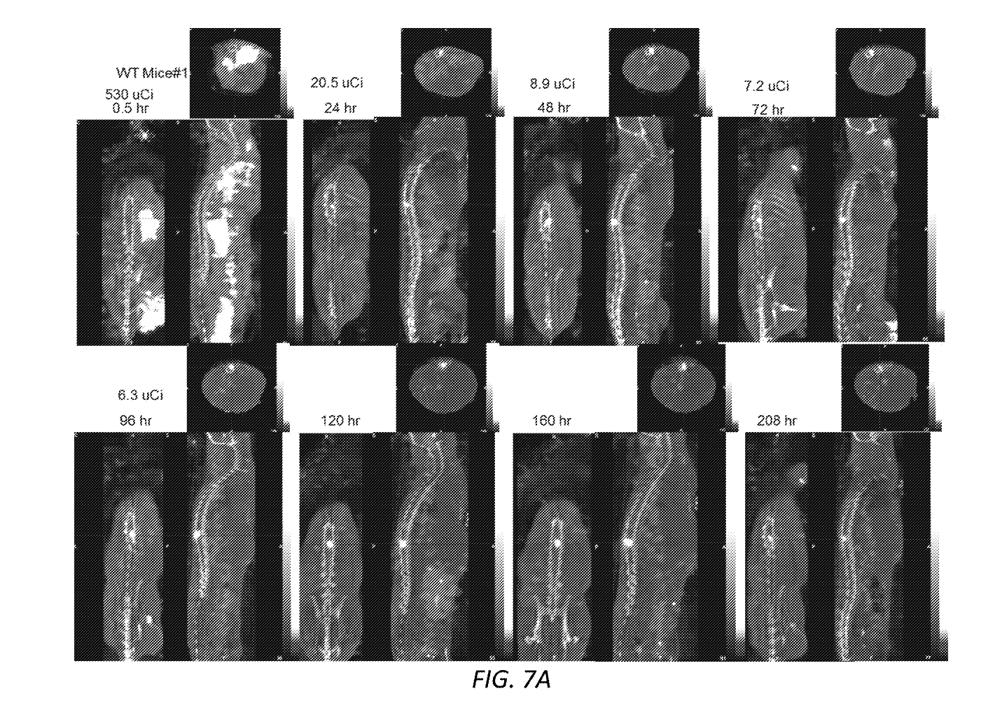

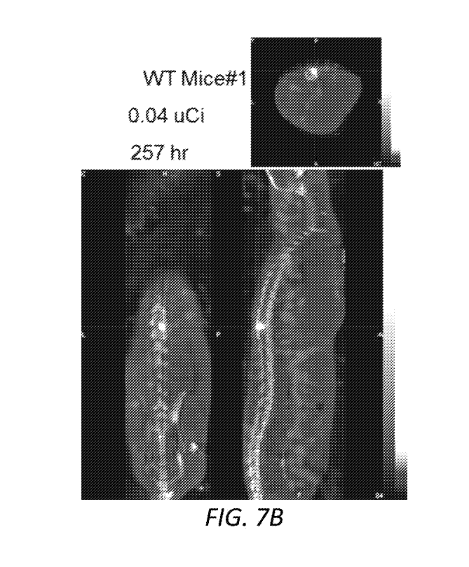

In normal, wildtype mice, when TTC-Indium¹¹¹ (FIG. 6) or TTC-I¹²⁵ (FIG. 7) was injected into the gastrocnemius muscle it was transported to the corresponding motor neurons at the L5 level in the spinal cord. The uptake had a characteristic time course for delivery (FIG. 8).

In both instances, the delivered TTC or tetanus toxoid was detectable for an extremely protracted period in the surrounding interneurons, suggesting that it persisted therein and was not further transported to the interneuronal cell body or via further trans-synaptic transport to other neurons. The rate of uptake of the labeled TTC from muscle and its arrival time in the motor neuron cell body reflected several properties of the neuromuscular system: (a) the integrity of the neuromuscular junction; (b) the rate of axonal transport; and (c) the trafficking of the TTC or toxoid within the motor neuron and surrounding interneurons. It is for this reason that we describe the final transport parameter as “net axonal transport.”

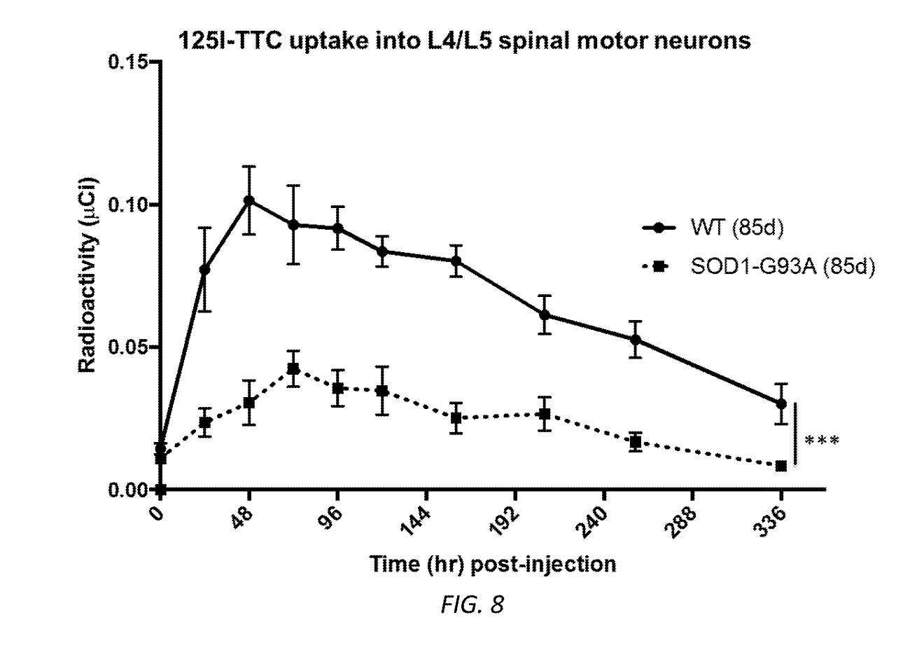

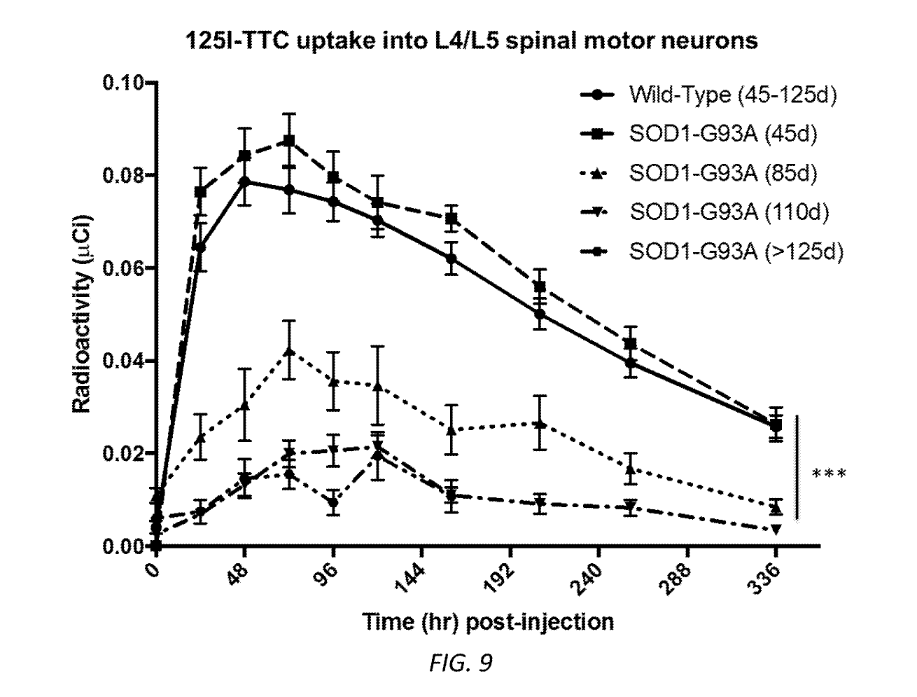

We also investigated the uptake of TTC-I¹²⁵ in a mouse model of amyotrophic lateral sclerosis (ALS, also known at Lou Gehrig's disease) that resulted from transgenic expression of mutant (G93A) superoxide dismutase. When TTC-I¹²⁵ was injected into the gastrocnemius muscle of 90 day old ALS mice, the level of TTC-I¹²⁵ uptake was reduced compared to comparable uptake in WT mice (FIGS. 8, 9).

In parallel studies, experiments were performed to determine if tetanus toxoid that was purified and used in the preparation of human diphtheria-tetanus-polio vaccinations might substitute for TTC in these experiments. Whereas TTC is a non-toxic, non-catalytic 450 amino acid fragment of the full TeNT molecule, tetanus toxoid is a preparation of the full TeNT toxin that has been inactivated by treatment with formalin. In pilot runs, we found that in vivo tetanus toxoid had lost most (but not all) of its ability to achieve low level binding to tetanus receptors in cell in vitro.

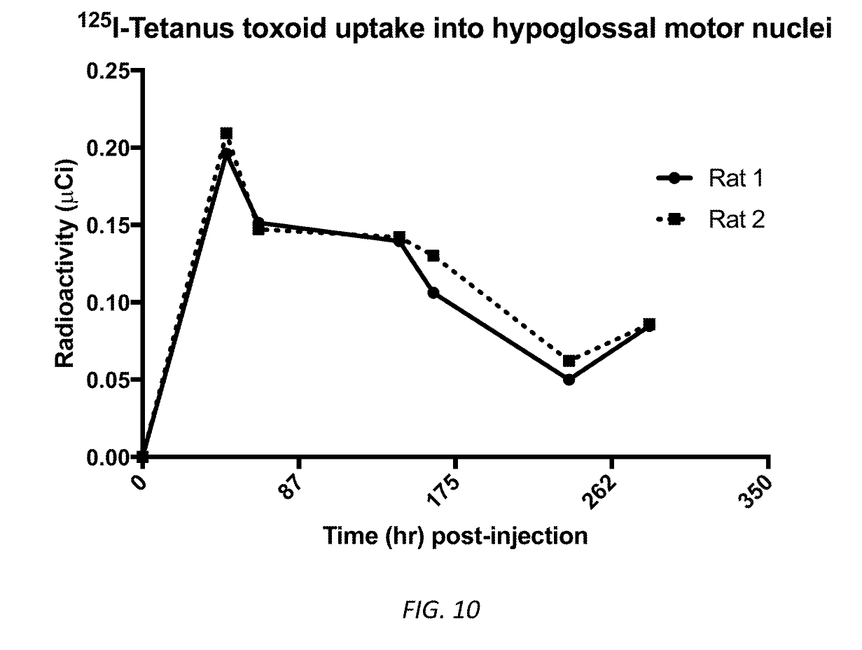

When tetanus toxoid was labeled with I¹²⁵, it (like TTC) could be tracked from skeletal muscle to the motor neurons, in experiments in which we performed injections into the rat tongue and tracked transport to bulbar motor neurons (FIG. 10). In a comparison of using I¹²⁵-TTC vs I¹²⁵-tetanus toxoid, the peak radioactivity of tetanus toxoid was around 0.2 μCi (at 47 hr post-injection; FIG. 10); the peak radioactivity of TTC is around 0.4 μCi (FIG. 10). Since the molar ratio of I¹²⁵:tetanus toxoid was around 2:1 while the molar ratio of I¹²⁵:TTC was around 1:1, the tetanus toxoid transport efficiency may be one quarter that of TTC, indicating that TTC is a better choice for these methods.

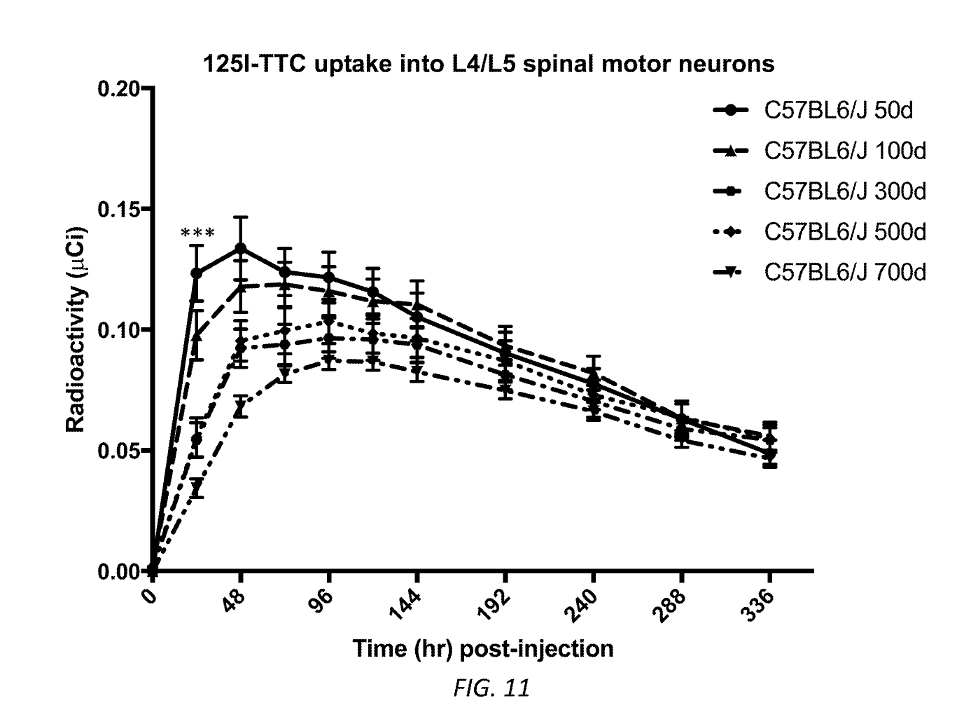

To examine the effects of aging on net axonal transport, transport curves (TTC uptake) were determined for WT mice of the following ages: 50, 100, 300, 500 and 700 days. As shown in FIG. 11, in WT mice, aging was associated with a reduction in the peak net axonal transport and a progressive delay in the time to peak.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of diagnosing pathology in motor neurons in a subject, the method comprising:
locally administering to a muscle of the subject a radiolabeled agent comprising tetanus toxic C fragment;
obtaining a first image of the agent in the subject prior to the muscle injection at a first time point;
obtaining at least a second image of the agent in the subject at a subsequent time point, and optionally multiple additional images at later time points;
determining at each time point after injection the total amount of the agent that is taken up into the motor neurons in the pool that innervates the injected muscle, to determine net total transport at that time point; and
using the net total transport at the time points to calculate the rate of uptake, to determine net total axonal transport rate; and
diagnosing the subject as having a motor neuron pathology, when the net total transport and net axonal transport rate are reduced relative to reference net total transport and net axonal transport rate.

2. The method of claim 1, further comprising administering a treatment for a motor neuron disease to the subject.

3. The method of claim 2, wherein the treatment is administration of riluzole, radicut, Edaravone, or inosine.

4. The method of claim 2, wherein the treatment is administration of stem cells.

5. A method of monitoring a motor neuron pathology in a subject, the method comprising:
determining net total transport and/or net axonal transport rate in the subject at a first time point;
determining net total transport and/or net axonal transport rate in the subject at a second time point;
comparing net total transport and/or net axonal transport rate in the subject at the first and second time points;
wherein a reduction in net total transport or net total axonal transport rate indicates that the disease has worsened or progressed; an increase in net total transport or net total axonal transport rate indicates that the disease has improved or regressed; and no change in net total transport or net total axonal transport rate indicates that the disease has neither worsened nor improved;
wherein determining net total transport and/or net axonal transport rate in the subject comprises locally administering to a muscle of the subject a radiolabeled agent comprising tetanus toxic C fragment;
obtaining a first image of the agent in the subject prior to the muscle injection at a first time point;
obtaining a second image of the agent in the subject at one or more subsequent time points;
determining at each time point after injection the total amount of the agent that is taken up into the motor neurons in the pool that innervates the injected muscle, to determine net total transport at that time point; and optionally
using the net total transport at the time points to calculate the rate of uptake, to determine net total axonal transport rate.

6. The method of claim 5, wherein a therapy was administered to the subject between the first and the later time points, and a reduction in net total transport and/or net axonal transport rate indicates that the therapy has had no effect; an increase in net total transport and/or net axonal transport rate indicates that the therapy is effective; and no change indicates that the therapy has stabilized the disease in a progressing subject or neither worsened nor improved the disease in a non-progressing subject.

7. The method of claim 1, wherein the imaging is performed using computed tomography (CT), magnetic resonance/nuclear magnetic resonance imaging (MRI/NMR), Single photon emission computed tomography (SPECT) or positron emission computed tomography (PET).

8. The method of claim 7, wherein the imaging is performed using an agent labeled with 64Cu, 67Ga, 86Y, 124I, 111In, 89Zr, or 99mTc.

9. The method of claim 1, wherein the radiolabeled agent comprising tetanus toxic C fragment comprises SEQ ID NO:2.

10. The method of claim 1, wherein the subject has, is suspected of having, or has been diagnosed with amyotrophic lateral sclerosis (ALS), peripheral neuropathy, or motor neuropathy, or has had a traumatic injury to a nerve.

11. The method of claim 5, wherein the imaging is performed using computed tomography (CT), magnetic resonance/nuclear magnetic resonance imaging (MRI/NMR), Single photon emission computed tomography (SPECT) or positron emission computed tomography (PET).

12. The method of claim 11, wherein the imaging is performed using an agent labeled with 64Cu, 67Ga, 86Y, 124I, 111In, 89Zr, or 99mTc.

13. The method of claim 5, wherein the radiolabeled agent comprising tetanus toxic C fragment comprises SEQ ID NO:2.

14. The method of claim 5, wherein the subject has, is suspected of having, or has been diagnosed with amyotrophic lateral sclerosis (ALS), peripheral neuropathy, or motor neuropathy, or has had a traumatic injury to a nerve.
